(19) United States
(12) Patent Application Publication
(10) Pub. No.: US 2018/0064757 A1
(43) Pub. Date: Mar. 8, 2018

(54) FORMULATION FOR PREVENTING OR TREATING DENTIN-ASSOCIATED SYMPTOMS OR DISEASES, AND METHOD USING THE SAME

(71) Applicant: Sancastle Worldwide Corporation, Taipei City (TW)

(72) Inventors: Chun-Pin Lin, Taipei City (TW); Hong-Ping Lin, Taipei City (TW); Wei-Yen Yeh, Taipei City (TW)

(21) Appl. No.: 15/259,862

(22) Filed: Sep. 8, 2016

(51) Int. Cl. A61K 33/42; A61K 33/06; A61K 33/00; A61K 33/30; A61K 33/24

(52) U.S. Cl. CPC A61K 33/42; A61K 33/06; A61K 33/24; A61K 33/30; A61K 33/00

(57) ABSTRACT

Provided is a non-aqueous formulation for oral teeth, which includes a source of a metal ion and a source of a phosphate ion. The metal ion is chosen from alkaline earth metals, Zn, Zr or any combination thereof, and a molar ratio of the metal ion to the phosphate ion in the formulation is between about 0.01 and about 1.0. The non-aqueous formulation can provide a therapeutic effect.

### BACKGROUND

### 1. Technical Field

The present disclosure relates to a formulation for use in oral teeth, and more particularly relates to a formulation for propylaxising and treating dentin-associated diseases or symptoms.

### 2. Description of Associated Art

Dentin, which is also known as “dentine,” is a tissue constituting the tooth matrix, and located between dental enamel and dental pulp. Dentin is composed of 70% of inorganic substance, 20% of organic substance and 10% of water. The hardness of dentin is lower than that of dental enamel, but higher than that of cementum. Dentinal tubules throughout the dentin are arranged radially from the surface of the dental pulp towards the dental enamel. The dentinal tubules are wider at the ends near the dental pulp. The closer toward the surface the dentinal tubules with branches therebetween are, the narrower they are.

Common dentin-associated diseases or symptoms, which cause pain, include dental caries, tooth wearing, enamel loss and dentin hypersensitivity etc.

Dentin hypersensitivity is sometimes referred to as “tooth hypersensitivity” and “hypersensitive dentine.” Currently, various types of products or methods for relieving dentin hypersensitivity have been developed. However, up to now, none of the products or methods can provide a rapid and long-term relieving effect.

Generally, the clinical methods for treating dentin hypersensitivity can fall into the following two categories: (1) chemical desensitizing method; and (2) physical desensitizing method.

As to the chemical desensitizing methods, corticosteroids were used in the early years to suppress inflammation. However, such methods are ineffective. Further, protein precipitation, which is also categorized as a chemical desensitizing method, utilizes a chemical agent to coagulate and denature the proteins in the dentinal tubule. For example, a formulation containing silver nitrate, phenol, formaldehyde or strontium chloride is used to denature collagen, and then forms precipitates which block the openings of the dentinal tubules. However, such formulation stimulates dental pulp and gingival, and the relapse rate is extremely high. Further, silver nitrate dyes teeth black permanently.

Moreover, chemical desensitizing methods also include a treating method for paralyzing pulp nerves. For example, some commercially available desensitizing toothpastes use potassium nitrate to suppress the excitation of pulp nerves. However, the clinical cases have shown that the pain on a patient cannot be relieved until the desensitizing toothpaste is persistently used for two weeks, and the therapeutic effect can last for only several months. That is to say, the methods for paralyzing pulp nerves cannot provide rapid and long-term therapeutic effects. Also, the long-term use of potassium nitrate leads to disorders associated with the paralysis of pulp nerves.

On the other hand, as to the physical desensitizing methods, for example, a sealant for dentinal tubules is used to directly seal the openings of dentinal tubules. The sealant includes, for example, resins, glass ionomer cements or the like. For example, Jensen et al. (“A comparative study of two clinical techniques for treatment of root surface hypersensitivity,” Gen. Dent. 35:128-132) proposed a method for directly sealing the openings of dentinal tubules using a resin-type dentin bonding agent. Although this method can immediately relieve the pain caused by dentin hypersensitivity, it cannot provide a long-term therapeutic effect. More specifically, the clinical cases have shown that, after a 6-months treatment, the resin-type bonding agent detaches significantly from the surfaces of teeth. As to the glass ionomer cements, Low et al. (“The treatment of hypersensitive cervical abrasion cavities using ASAP cement,” J. Oral Rehabil. 8(1):81-9) used glass ionomer cements to treat dentin hypersensitivity in 1981. Although glass ionomer cements can provide therapeutic effects, this type of material will be removed by constantly brushing the tooth. Further, Hansen et al. (“Dentin hypersensitivity treated with a fluoride-containing varnish or a light-curd glass ionomer liner,” Scand. J. Dent. Res. 100(6):305-9) used resin-enhanced glass ionomer cements to treat dentin hypersensitivity, but still no long-term therapeutic effects were achieved.

Recently, formation of precipitates on enamel has been reported that can recover teeth enamel. Nevertheless, the precipitates can only be formed on the surface of teeth, and thus are easily detached from the tooth surface.

Accordingly, it is an urgent and important issue to provide a rapid and efficient effect to relieve dentin-associated symptoms and diseases.

### SUMMARY

In view of the foregoing, the present disclosure provides a non-aqueous formulation for oral teeth, comprising a source of a metal ion and a source of a phosphate ion. The metal ion is chosen from alkaline earth metals, Zn, Zr or any combination thereof, and a molar ratio of the metal ion to the phosphate ion in the formulation is between about 0.01 and about 1.0. The source of the phosphate ion of the non-aqueous formulation is a kind of soluble phosphates. As applying the formulation for oral teeth of the present disclosure to teeth with dentin-associated symptoms or diseases, the formulation of the present disclosure can rapidly and efficiently treat dentin-associated symptoms or diseases within hours, preferably minutes, by forming precipitates in the dentinal tubules, without the need to use any particulate carrier preformed with the source of metal ions for carrying the metal ions. In addition, the depths of the precipitates in the dentinal tubules are deep enough for the sealing effect of dentinal tubules to be maintained for a long period of time. The aforementioned precipitates are formed by the metal ion and the phosphate ion.

The present disclosure further provides a method for preventing or treating a dentin-associated symptom or disease, comprising administering the aforesaid non-aqueous formulation for oral teeth to the oral cavity of a subject.

In one embodiment, the dentine tubule-associated syndrome is dentin hypersensitivity, crack tooth syndrome, enamel loss, dentin loss or postoperative hypersensitivity. In another embodiment, the dentin-associated disease is dental caries, root caries, tooth fracture, root fracture, cervical abrasion, tooth wearing, root perforation, radicular cyst, apicitis, pulpitis, periapical periodontitis, pulp necrosis or dentin-associated pulp disease.

The present disclosure further provides a method for dental therapy, comprising: providing the aforesaid non-aqueous formulation; and administering the non-aqueous formulation to the oral cavity of a subject by smearing, pasting, attaching or brushing.

### BRIEF DESCRIPTION OF THE DRAWINGS

The present disclosure can be more fully understood by reading the following detailed description of the embodiments, with reference made to the accompanying drawings, wherein:

FIG. 1A shows an SEM observation of a dental test slice coated with the formulation comprising calcium ion and phosphate ion of an embodiment of the present disclosure;

FIG. 1B shows an SEM observation of a dental test slice coated with the formulation comprising calcium ion and phosphate ion of an embodiment of the present disclosure;

FIG. 1C shows an SEM observation of a dental test slice coated with the formulation comprising calcium ion and phosphate ion of an embodiment of the present disclosure;

FIG. 1D shows an SEM observation of a dental test slice coated with the formulation comprising calcium ion and phosphate ion of an embodiment of the present disclosure;

FIG. 1E shows an SEM observation of a dental test slice coated with the formulation comprising calcium ion and phosphate ion of an embodiment of the present disclosure;

FIG. 1F shows an SEM observation of a dental test slice coated with the formulation comprising calcium ion and phosphate ion of an embodiment of the present disclosure;

FIG. 2A shows an SEM observation of a dental test slice coated with the formulation comprising calcium ion and phosphate ion of an embodiment of the present disclosure;

FIG. 2B shows an SEM observation of a dental test slice coated with the formulation comprising calcium ion and phosphate ion of an embodiment of the present disclosure;

FIG. 2C shows an SEM observation of a dental test slice coated with the formulation comprising calcium ion and phosphate ion of an embodiment of the present disclosure;

FIG. 3A shows an SEM observation of a dental test slice coated with the formulation comprising calcium ion and phosphate ion of an embodiment of the present disclosure;

FIG. 3B shows an SEM observation of a dental test slice coated with the formulation comprising calcium ion and phosphate ion of an embodiment of the present disclosure;

FIG. 3C shows an SEM observation of a dental test slice coated with the formulation comprising calcium ion and phosphate ion of an embodiment of the present disclosure;

FIG. 3D shows an SEM observation of a dental test slice coated with the formulation comprising calcium ion and phosphate ion of an embodiment of the present disclosure;

FIG. 3E shows an SEM observation of a dental test slice coated with the formulation comprising calcium ion and phosphate ion of an embodiment of the present disclosure;

FIG. 4A shows an SEM observation of a dental test slice coated with the formulation comprising magnesium ion and phosphate ion of an embodiment of the present disclosure;

FIG. 4B shows an SEM observation of a dental test slice coated with the formulation comprising strontium ion and phosphate ion of an embodiment of the present disclosure;

FIG. 4C shows an SEM observation of a dental test slice coated with the formulation comprising zinc ion and phosphate ion of an embodiment of the present disclosure;

FIG. 4D shows an SEM observation of a dental test slice coated with the formulation comprising zirconium ion and phosphate ion of an embodiment of the present disclosure;

FIG. 5A shows an SEM observation of a dental test slice coated with the formulation comprising calcium ion, phosphate ion and zinc phosphate of an embodiment of the present disclosure;

FIG. 5B shows an SEM observation of a dental test slice coated with the formulation comprising calcium ion, phosphate ion and methyl cellulose of an embodiment of the present disclosure;

FIG. 5C shows an SEM observation of a dental test slice coated with the formulation comprising calcium ion, phosphate ion and carbomer of an embodiment of the present disclosure;

FIG. 5D shows an SEM observation of a dental test slice coated with the formulation comprising calcium ion, phosphate ion and titanium dioxide of an embodiment of the present disclosure;

FIG. 5E shows an SEM observation of a dental test slice coated with the formulation comprising calcium ion, phosphate ion and calcium phosphate of an embodiment of the present disclosure;

FIG. 6 shows an SEM observation of a dental test slice coated with the formulation in a form of orabase of an embodiment of the present disclosure;

FIG. 7A shows a High-performance Liquid Chromatography (HPLC) result of assay of betamethasone sodium phosphate in Standard 1;

FIG. 7B shows a HPLC result of assay of betamethasone sodium phosphate in an embodiment of the present disclosure;

FIG. 8A shows a HPLC result of assay of clindamycin phosphate in Standard 2;

FIG. 8B shows a HPLC result of assay of clindamycin phosphate in an embodiment of the present disclosure;

FIG. 9 shows an SEM observation of a dental test slice coated with the formulation comprising calcium ion, phosphate ion and betamethasone sodium phosphate of an embodiment of the present disclosure.

### DETAILED DESCRIPTION OF THE EMBODIMENTS

The following specific examples are used to exemplify the present disclosure. A person of ordinary skills in the art can conceive the other advantages of the present disclosure, based on the disclosure of the specification of the present disclosure. The present disclosure can also be implemented or applied as described in different specific examples. It is possible to modify and/or alter the above examples for carrying out this disclosure without contravening its spirit and scope, for different aspects and applications.

In order to solve the issues in prior art, the inventors accomplished a non-aqueous formulation for oral teeth after performing a variety of experiments. The formulation comprises a source of a metal ion and a source of a phosphate ion, wherein the metal ion is chosen from alkaline earth metals, Zn, Zr or any combination thereof, and a molar ratio of a metal ion to a phosphate ion in the formulation is between about 0.01 and about 1.0, and wherein the metal ion and the phosphate ion form a precipitate in dentinal tubules of the teeth.

In an embodiment of the formulation for oral teeth, a molar ratio of the metal ion to the phosphate ion in the formulation has a lower limit chosen from 0.01, 0.05, 0.1, 0.15, 0.2, 0.25, 0.3, 0.35, 0.4, 0.45 and 0.5 and an upper limit chosen from 1, 0.9, 0.8, 0.7 and 0.6. In an embodiment of the formulation for oral teeth, a molar ratio of the metal ion to the phosphate ion in the formulation is between about 0.1 to about 1. In an embodiment, the molar ratio of the metal ion to the phosphate ion in the formulation is between about 0.2 to about 1. In an embodiment, the molar ratio of the metal ion to the phosphate ion in the formulation is between about 0.2 to about 0.6.

In an embodiment of the formulation for oral teeth, the formulation is used in combination with water or saliva to form a mixture having a pH value between about 2.0 and about 6. In an embodiment, the pH of the mixture has a lower limit chosen from 2.0, 2.5, 3, 3.5 and 4 and an upper limit chosen from 6, 5.5 and 5. In an embodiment, the pH value of the mixture is between about 2.0 and about 5.5. In an embodiment, the pH value of the mixture is between about 2.0 and about 5.0. In an embodiment, the pH value of the mixture is between about 2.0 and about 4.0. In an embodiment, the pH value of the mixture is between about 3.0 and about 4.0. In an embodiment, the pH value of the mixture is between about 4.0 and about 6.0.

In an embodiment of the formulation for oral teeth, the metal ion may be selected from the group consisting of magnesium ion, calcium ion, strontium ion and barium ion.

In an embodiment of the formulation for oral teeth, the source of the metal ion may be selected from the group consisting of carbonates, acetates, lactates, citrates, chlorides, oxides, nitrates and hydroxides.

In an embodiment of the formulation for oral teeth, the formulation of the present disclosure is preferably non-aqueous. As used herein, the term “non-aqueous” means that the formulation does not include water in such an amount that will prematurely trigger the reaction of the component(s) in the formulation, and/or reduce the stability of the formulation. In an embodiment, the formulation of the present disclosure includes either no water or only traces of water from, for example, salts with water of hydration. In another embodiment, the individual components of the non-aqueous formulation may contain limited amounts of water as long as the overall formulation remains substantially free of water. Thus, in certain embodiments, no water is added to the formulation prior to use.

In an embodiment of the formulation for oral teeth, the source of the phosphate ion may be at least one selected from the group consisting of disodium hydrogen phosphate, dipotassium hydrogen phosphate, lithium dihydrogen phosphate, sodium dihydrogen phosphate, potassium dihydrogen phosphate, trisodium phosphate, tripotassium phosphate, ammonium phosphate, and a phosphate-containing drug.

In an embodiment of the formulation for oral teeth, the phosphate-containing drug may be selected from the group consisting of tetracycline phosphate complex, oleadomycin phosphate, codeine phosphate, estramustine phosphate, primaquine phosphate, dimemorfan phosphate, pyrldoxal phosphate, pyridoxal phosphate, piperazine phosphate, clindamycin phosphate, sodium phosphate, dexamethasone sodium phosphate, oseltamivir phosphate, benproperine phosphate, prednisolone sodium phosphate, betamethasone sodium phosphate, chloroquine phosphate, disopyramide phosphate, etoposide phosphate, fludarabine phosphate, histamine phosphate, hydrocortisone sodium phosphate, sodium biphosphate, ruxolitinib phosphate, sitagliptin phosphate, anileridine phosphate, sonidegib phosphate, oritavancin diphosphate, tedizolid phosphate, antazoline phosphate, estramustine phosphate, codeine phosphate, toceranib phosphate and chromic phosphate P32.

In an embodiment of the formulation for oral teeth, the formulation further comprises an additive, wherein the additive may be a thickening agent, an adhesive, an excipient, a stabilizer, an emulsifier, a humectant, or a combination thereof.

In an embodiment of the formulation for oral teeth, the thickening agent may be selected from the group consisting of methyl cellulose, hydroxyethyl cellulose, carbomer, titanium dioxide, zinc phosphate, zinc oxide, silicon dioxide, silicoaluminate, aluminum oxide and calcium phosphate.

In an embodiment of the formulation for oral teeth, the adhesive may be selected from the group consisting of acacia, alginate, alginic acid, candelilla wax, carnuba wax, corn, starch, copolyvidone, ethyl cellulose, gelatin, glyceryl behenate, hydroxyl propyl cellulose, hydroxyl propyl methyl cellulose, hypromellose, lactose hydrous, lactose anhydrous, lactose monohydrate, lactose spray dried, methyl cellulose, povidone, polyvinylpyrrolidone, polyethylene oxide, potato starch, starch pregelatinized, starch, sodium starch and sodium carboxy methyl cellulose.

In an embodiment of the formulation for oral teeth, the excipient may be pectin, eudragit or a combination thereof.

In an embodiment of the formulation for oral teeth, the formulation may be in the form of a powder, a paste, a flake, a gel, a soft gel, a gum, a semi-solid, a slurry, a patch, an emulsion, a glue, a buccal tablet, a pill, a film, a cream, an aerosol, or an orabase.

According to another aspect of the present disclosure, the present disclosure further provides a method for preventing or treating a dentin-associated symptom or disease, comprising the step of administering the aforesaid non-aqueous formulation of the present disclosure to the oral cavity of a subject. More specifically, the dentin-associated symptoms which can be prevented or treated by the method of the present disclosure are not limited, and may include dentin hypersensitivity, crack tooth syndrome, enamel loss, dentin loss, and postoperative hypersensitivity. Enamel loss or dentin loss is generally caused by corrosion, abrasion, wearing or cracking to the teeth. Postoperative hypersensitivity generally occurs after dental operations such as teeth bleaching, prosthodontic and restoration.

Further, the diseases which can be prevented or treated by the method of the present disclosure are not limited, and preferably include dental caries, root caries, tooth fracture, root fracture, cervical abrasion, tooth wearing, root perforation, radicular cyst, apicitis, pulpitis, periapical periodontitis, pulp necrosis, and dentin-associated pulp disease.

According to another aspect of the present disclosure, the present disclosure provides a method for dental therapy, comprising the steps of: providing the aforesaid non-aqueous formulation; and administering the non-aqueous formulation to the oral cavity of a subject by smearing, pasting, attaching or brushing.

### EXAMPLE

### Example 1: Preparation of Formulations Having Different Molar Ratios of Calcium Ion to Phosphate Ion with a pH Value Between 2 and 3

KH₂PO₄ and K₂HPO₄ were ground and mixed well with SiO₂ as a thickening agent, and then CaCl₂ powder was added thereto and mixed well to form a solid material. The solid material was then mixed with water to obtain Samples 1-6. The amounts of SiO₂, KH₂PO₄, K₂HPO₄ and CaCl₂ powder in Samples 1-6, as well as the molar ratios of calcium ion to phosphate ion (Ca/P) and the pH value of Samples 1-6 were shown in the following Table 1.

TABLE 1
Contents, Ca/P ratios and pH values of Samples 1-6

| | | | | CaCl₂ | | | |
|---|---|---|---|---|---|---|---|
| | SiO₂ | KH₂PO₄ | K₂HPO₄ | powder | Water | Ca/P | pH |
| | (g) | (g) | (g) | (g) | (g) | ratio | value |
| Sample 1 | 0.1484 | 0.42 | 0 | 0.0685 | 0.83 | 0.2 | 2 |
| Sample 2 | 0.1484 | 0.42 | 0 | 0.2056 | 0.83 | 0.6 | 2 |
| Sample 3 | 0.1484 | 0.42 | 0 | 0.3425 | 0.83 | 1 | 2 |
| Sample 4 | 0.1484 | 0.3675 | 0.06725 | 0.0685 | 0.83 | 0.2 | 3 |
| Sample 5 | 0.1484 | 0.21 | 0.269 | 0.2056 | 0.83 | 0.6 | 3 |
| Sample 6 | 0.1484 | 0.21 | 0.269 | 0.3425 | 0.83 | 1 | 2.7 |

### Example 2: Preparation of Formulations Containing Molar Ratio of Calcium Ion to Phosphate Ion of 0.2 or 0.6 with Different pH Values

Preparation of the formulations having molar ratio of calcium ion to phosphate ion of 0.2 or 0.6 with different pH values was the same as that described in Example 1. The amounts of SiO₂, KH₂PO₄, K₂HPO₄ and CaCl₂ powder in Samples 7-9, as well as the molar ratio of calcium ion to phosphate ion and the pH value of Samples 7-9 were shown in the following Table 2.

TABLE 2
Contents, Ca/P ratios and pH values of Samples 7-9

| | SiO₂ | KH₂PO₄ | K₂HPO₄ | CaCl₂ | Water | Ca/P | pH |
|---|---|---|---|---|---|---|---|
| | (g) | (g) | (g) | powder (g) | (g) | ratio | value |
| Sample 7 | 0.1484 | 0.3413 | 0.101 | 0.0685 | 0.83 | 0.2 | 3.5 |
| Sample 8 | 0.1484 | 0.315 | 0.1345 | 0.0685 | 0.83 | 0.2 | 4 |
| Sample 9 | 0.1484 | 0.2625 | 0.2018 | 0.0685 | 0.83 | 0.2 | 5 |

### Example 3: Preparation of Formulations Containing Magnesium Ion, Strontium Ion, Zinc Ion or Zirconium Ion

In addition to calcium ion, the other ions of alkaline earth metals such as Mg and Sr would create precipitation with phosphate ion. Further, in dentistry, Zn and Zr are commonly used and have low toxicity for human, and they could also create precipitation with phosphate ion. Thus, the inventors additionally used the magnesium ion, strontium ion, zinc ion and zirconium ion to accomplish the present disclosure.

Preparation of the formulations having magnesium ion, strontium ion, zinc ion or zirconium ion was the same as that described in Example 1. The amounts of SiO₂, KH₂PO₄, K₂HPO₄ and MgCl₂ powder, SrCl₂ powder, ZnSO₄.7H₂O powder or ZrOCL₂ powder in Samples 10-13, as well as the molar ratio of metal ion to phosphate ion (i.e., Mg/P, Sr/P, Zn/P or Zr/P) and the pH values of Samples 10-13 were shown in the following Table 3.

TABLE 3
Contents, metal ion/P ratios and pH values of Samples 10-13

| | | | | | | | Metal | |
|---|---|---|---|---|---|---|---|---|
| | SiO₂ | KH₂PO₄ | K₂HPO₄ | | | Water | ion/P | pH |
| | (g) | (g) | (g) | Metal ion powder (g) | | (g) | ratio | value |
| Sample 10 | 0.1484 | 0.42 | 0 | MgCl₂ | 0.1255 | 0.83 | 0.2 | 3 |
| Sample 11 | 0.1484 | 0.42 | 0 | SrCl₂ | 0.1646 | 0.75 | 0.2 | 2 |
| Sample 12 | 0.1484 | 0.42 | 0 | ZnSO₄ · 7H₂O | 0.178 | 0.83 | 0.2 | 3 |
| Sample 13 | 0.1484 | 0.42 | 0 | ZrOCL₂ · 8H₂O | 0.199 | 0.83 | 0.2 | 2 |

### Example 4: Preparation of Formulations Containing Calcium Ion, Phosphate Ion and Different Kinds of Thickening Agents

Preparation of the formulations respectively having different kinds of thickening agents was the same as that described in Example 1. The amounts of KH₂PO₄, K₂HPO₄, CaCl₂ powder and the thickening agent (zinc phosphate, methyl cellulose, carbomer, titanium dioxide or calcium phosphate) in Samples 14-18, as well as the molar ratio of calcium ion to phosphate ion and the pH value of Samples 14-18 were shown in the following Table 4.

TABLE 4
Contents, Ca/P ratios and pH values of Samples 14-18

| | | | | | CaCl₂ | | | |
|---|---|---|---|---|---|---|---|---|
| | Thickening agent | | KH₂PO₄ | K₂HPO₄ | powder | Water | | pH |
| | (g) | | (g) | (g) | (g) | (g) | Ca/P | value |
| Sample 14 | zinc | 2.2065 | 0.42 | 0 | 0.0685 | 0.83 | 0.2 | 3 |
| | phosphate | | | | | | | |
| Sample 15 | methyl | 0.3 | 0.42 | 0 | 0.0685 | 0.83 | 0.2 | 3 |
| | cellulose | | | | | | | |
| Sample 16 | carbomer | 0.15 | 0.42 | 0 | 0.0685 | 0.83 | 0.2 | 2 |
| Sample 17 | titanium | 0.3 | 0.42 | 0 | 0.0685 | 0.83 | 0.2 | 2 |
| | dioxide | | | | | | | |
| Sample 18 | calcium | 0.6 | 0.42 | 0 | 0.0685 | 0.83 | 0.2 | 3 |
| | phosphate | | | | | | | |

### Example 5: Preparation of Formulation in a Form of Orabase

Preparation of the formulation in a form of orabase was similar to that described in Example 1 except that the solid material containing CaCl₂ and KH₂PO₄ was mixed with water and then incorporated into to Carboxe orabase, so as to obtain Sample 19. Carboxe orabase is a hydrophobic substance and contains Carbenoxolone. If the orabase is applied to oral cavity and mixed with saliva, it will become thick and release hydrophilic substances therein, thereby treating mouth wounds.

The amounts of CaCl₂ and KH₂PO₄ powder and Carboxe orabase in Sample 19, as well as the molar ratio of calcium ion to phosphate ion and the pH value of Sample 19 were shown in the following Table 5.

TABLE 5
Contents of Sample 19

| | | CaCl₂ | Carboxe | | | |
|---|---|---|---|---|---|---|
| | KH₂PO₄ | powder | orabase | Water | Ca/P | pH |
| | (g) | (g) | (g) | (g) | ratio | value |
| Sample 19 | 0.42 | 0.0685 | 0.495 | 0.83 | 0.2 | 2-3 |

### Test Example 1: Sealing Effect of Dentinal Tubules In Vitro

Twenty premolars and molars, provided with complete crowns having no caries and no fillers, just removed from a human were collected.

An ultrasonic dental scaler (Sonicflex 2000, Kayo Co Biberbach, Germany) was used to remove dental calculus and periodontal tissues from the premolars and molars. Then, the premolars and molars were stocked in 4° C. distilled water, so as to maintain the freshness of the dentin.

Before applying the formulation, the teeth were taken out of the water, and the enamel at the occlusion site was removed in a horizontal direction using a low speed saw (Isomet low speed saw, Buehler, LTD.), and incised at a distance of 1.5 mm along the direction of the neck to obtain a specimen of dentin. Then, a tapered fissure bur (1961 tapered fissure bur) was used to create a groove on the back of the experimental area of each of the specimens, to guide the direction of future incision of the specimens. Thirty-seven point five percent of phosphoric acid as gel etchant (Kerr Co USA) was used to acid etch the specimens up to 40 seconds. Then, a large amount of distilled water was used to wash the coating layer, and the surfaces of the specimens were blow-dried.

The formulations of Samples 1-6 recited in Example 1, Samples 7-9 recited in Example 2, Samples 10-13 recited in Example 3, Samples 14-18 recited in Example 4 and Sample 19 recited in Example 5 were used to coat the specimens, respectively, by the following approaches.

To examine the sealing effect on dentin of the formulation of Sample 1 recited in Example 1, the formulation was applied on the surfaces of tooth samples, allowing it to react for 10 minutes, 10 minutes with 3 minutes/3 minutes/4 minutes intervals, 30 minutes with 6 times 5 minutes intervals, 0.5 day, 1 day and 2 days.

Then, a field emission scanning electronic microscope (SEM; Field Emission Scanning Electronic Microscope Hitachi S-800, Hitachi Co., Tokyo, Japan) was used to observe the depth of the precipitates in the dentinal tubules in each of the samples.

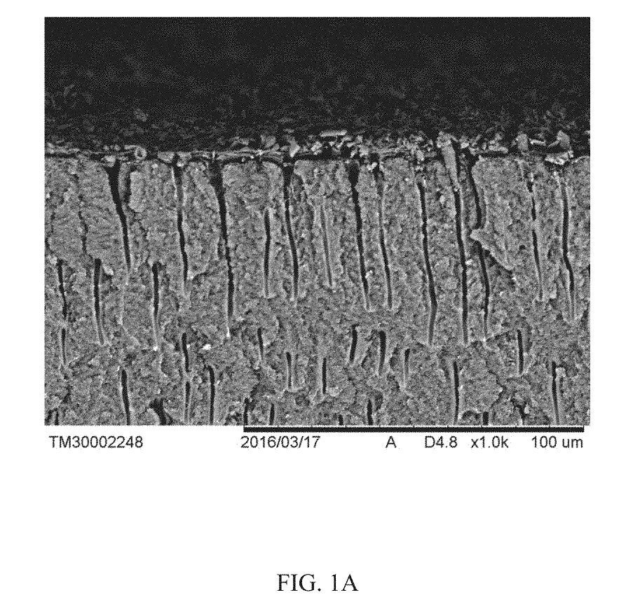

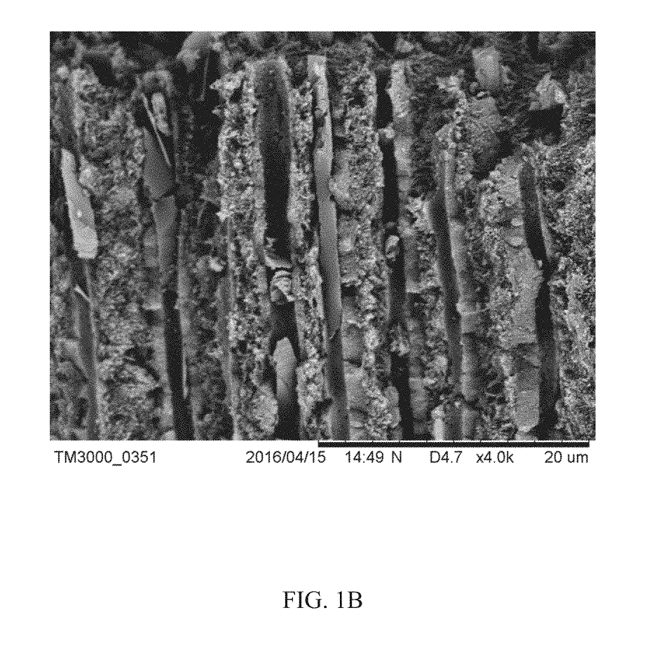

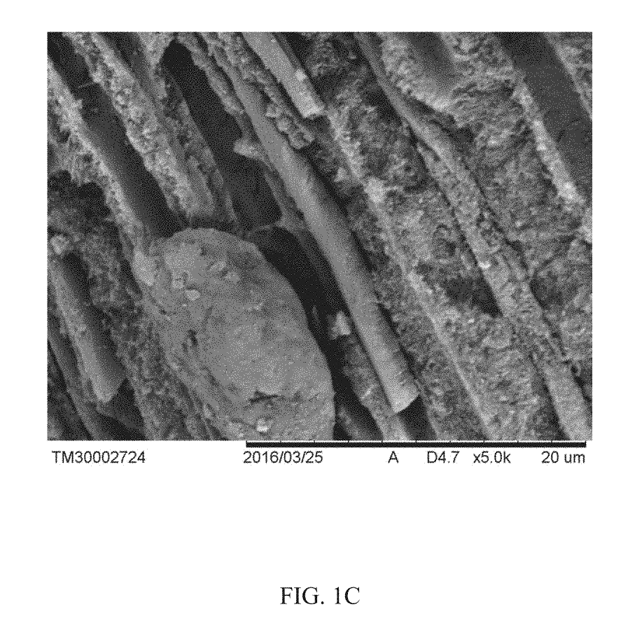

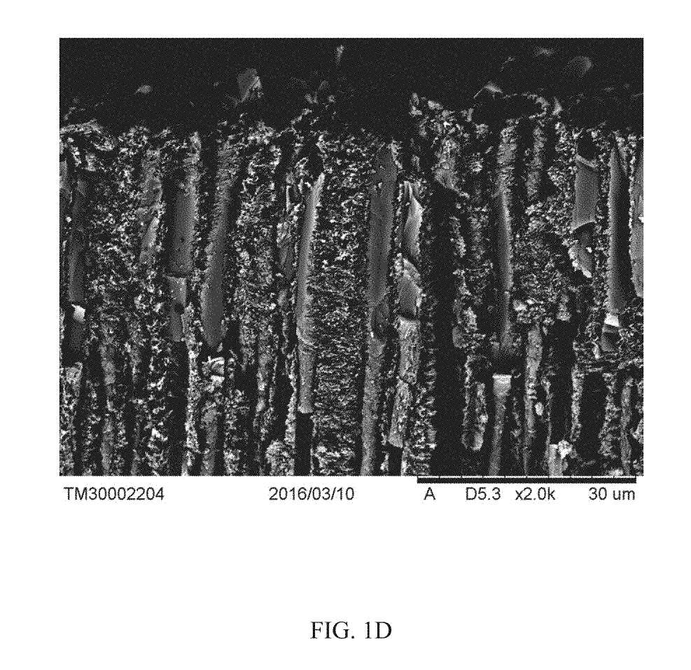

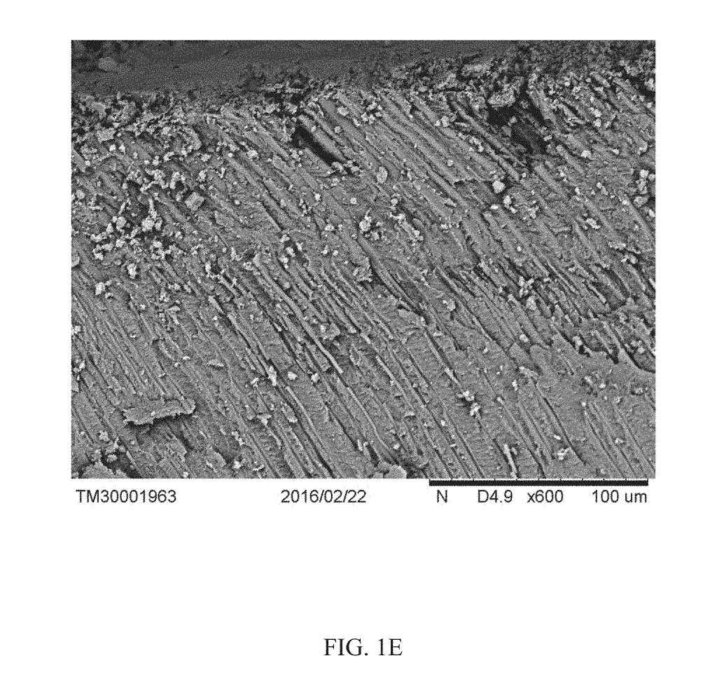

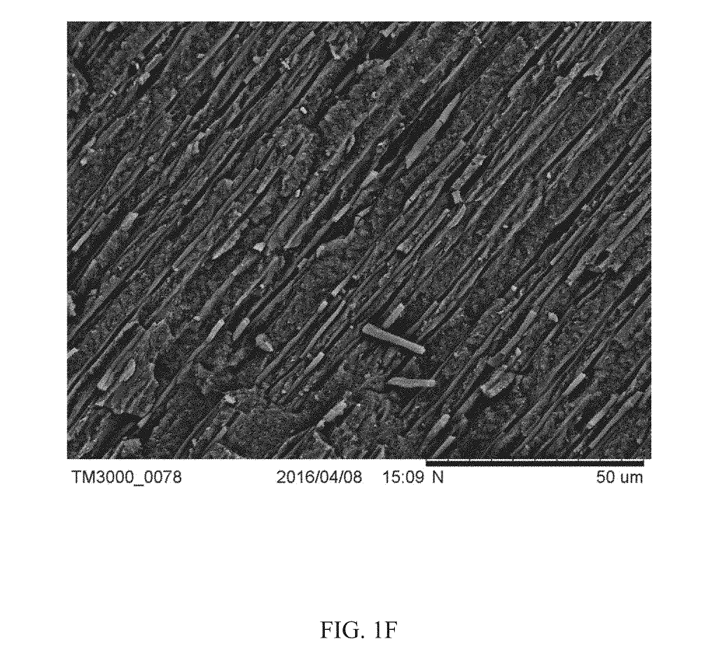

FIGS. 1A-1F respectively showed the SEM observations of each of the specimens reacted for 10 minutes, 10 minutes with 3 minutes/3 minutes/4 minutes intervals, 30 minutes with 6 times 5 minutes intervals, 0.5 day, 1 day and 2 days. The results showed that the formulation having a molar ratio of calcium ion to phosphate ion of 0.2 with a pH value of 2 could effectively occlude dentinal tubes within 10 minutes. The depths of the precipitates in the dentinal tubules of Sample 1 after occluding for different period of time were shown in Table 6. The results showed that the formulation provided better sealing effect on dentin by increasing reaction time.

TABLE 6
The depths of the precipitates in the dentinal tubules of Sample 1 with
different reaction time

| | Depth (μm) | Duration (hour) |
|---|---|---|
| Sample 1 | 36 | 0.1667 |
| Sample 1 | 56 | 0.5 |
| Sample 1 | 114 | 12 |
| Sample 1 | 176 | 24 |
| Sample 1 | 267 | 48 |

To examine the sealing effect of the formulations of Samples 1-3 with different molar ratios of calcium ion to phosphate ion and a pH value of 2 recited in Example 1, each of the formulation of Samples 1-3 was applied on the surfaces of tooth samples, allowing it to react for 30 minutes. Then, the depth of the precipitates in the dentinal tubules of each of the specimens was observed by SEM.

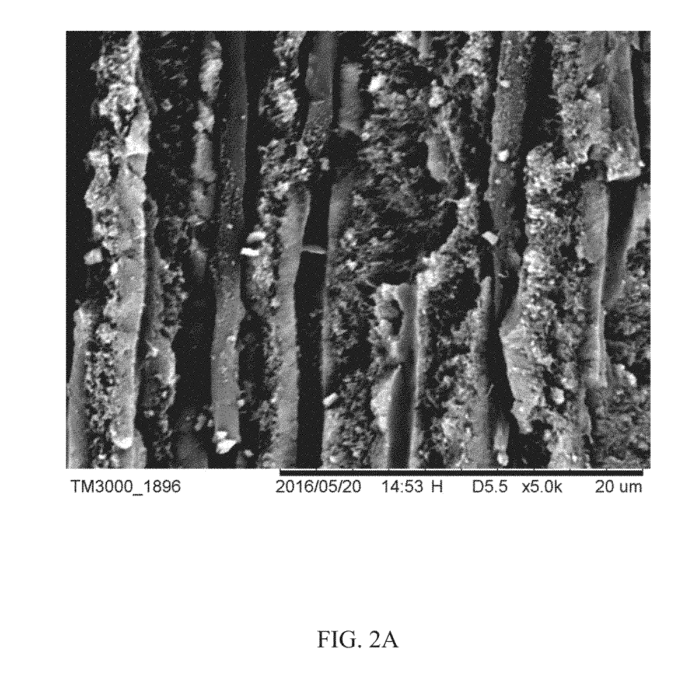

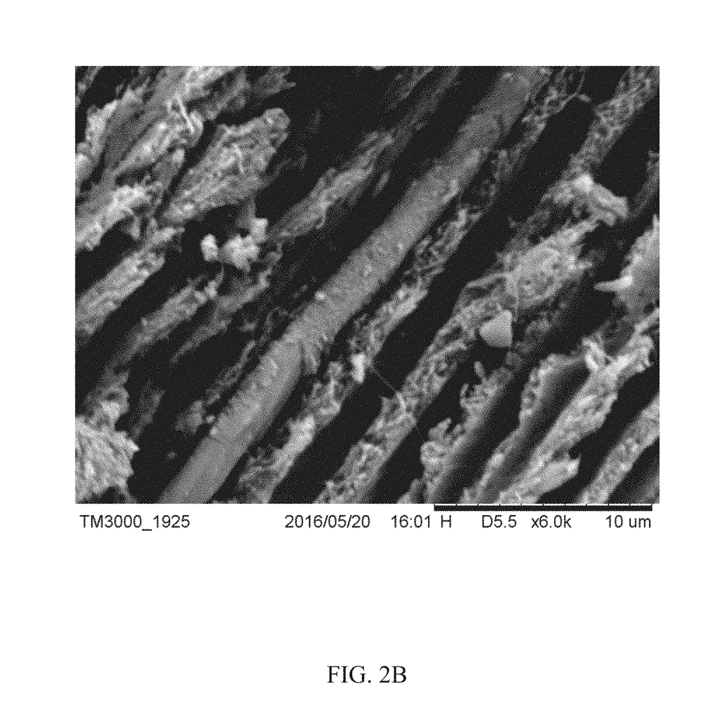

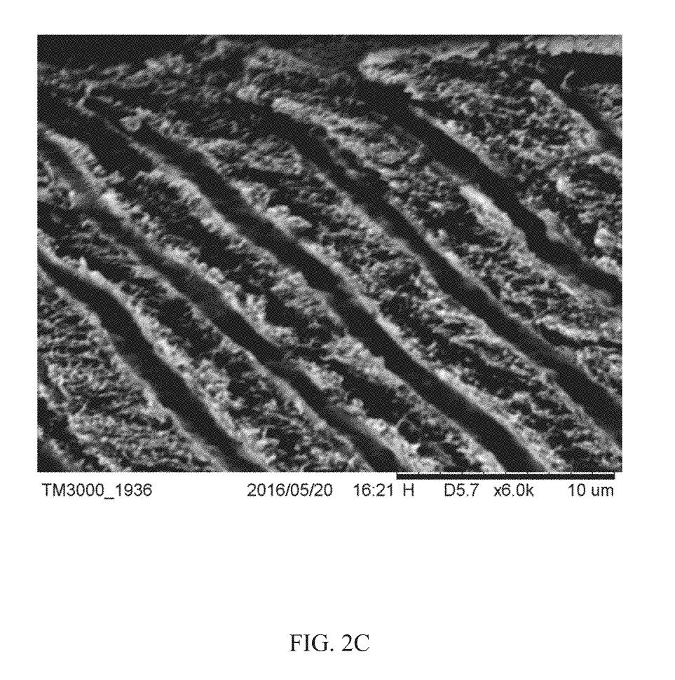

FIGS. 2A-2C respectively showed the SEM observations of each of the specimens of Samples 1-3, and the depths of the precipitates in the dentinal tubules of Samples 1-3 after occluding were shown in Table 7. The results showed that the sealing effect of the formulation having a molar ratio of calcium ion to phosphate ion of 0.2 to 0.6 was better than that of the formulation having a molar ratio of calcium ion to phosphate ion of 1.

TABLE 7
The depths of the precipitates in the dentinal tubules of Samples 1-3

| | depth (μm) |
|---|---|
| Sample 1 | 56 |
| Sample 2 | 40 |
| Sample 3 | X |

To examine the sealing effect of the formulations of Samples 7-9 with molar ratio of calcium ion to phosphate ion of 0.2 and pH values of 3.5, 4 and 5 on dentin, each of the formulation of Samples 7-9 was applied on the surfaces of tooth samples, allowing it to react for 1 hour or 1 day. Then, the depth of the precipitates in the dentinal tubules in each of the specimens was observed by SEM.

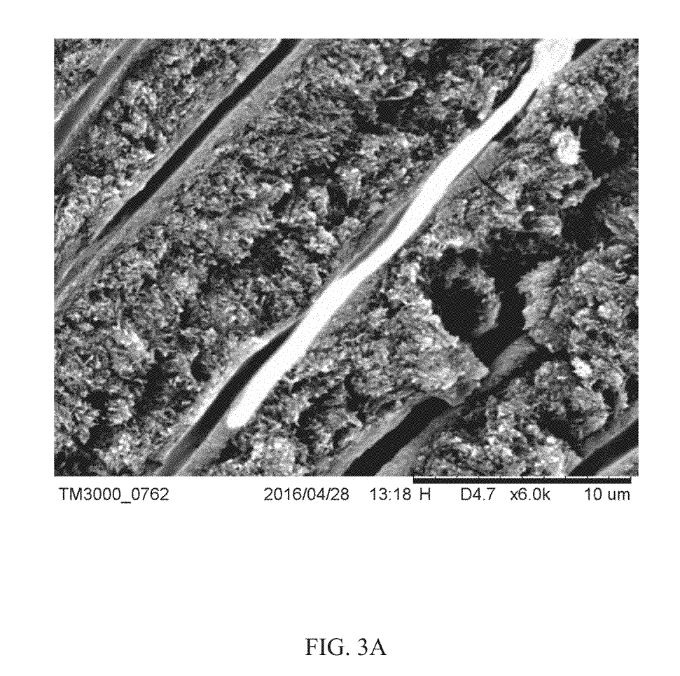

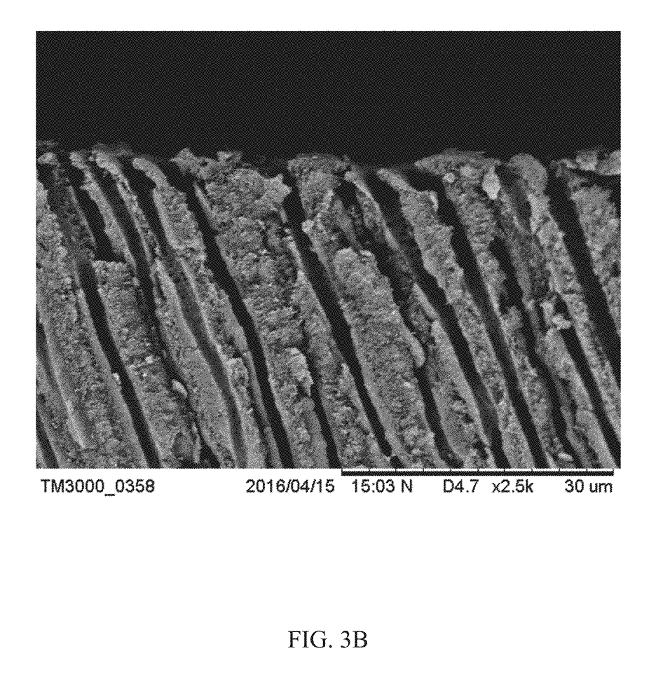

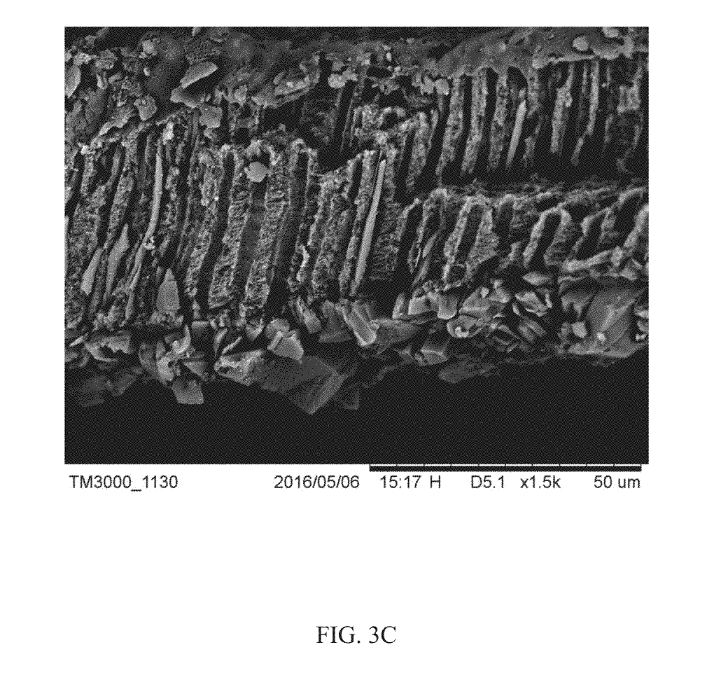

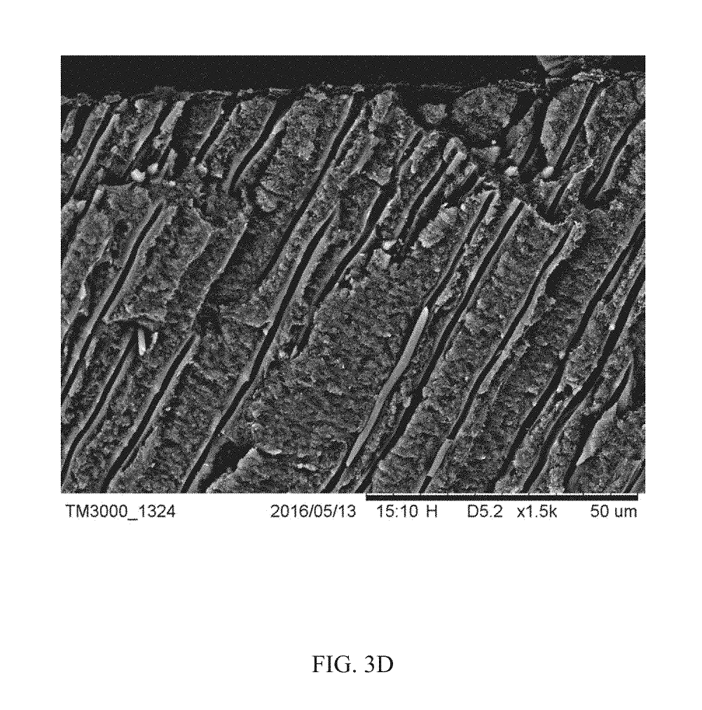

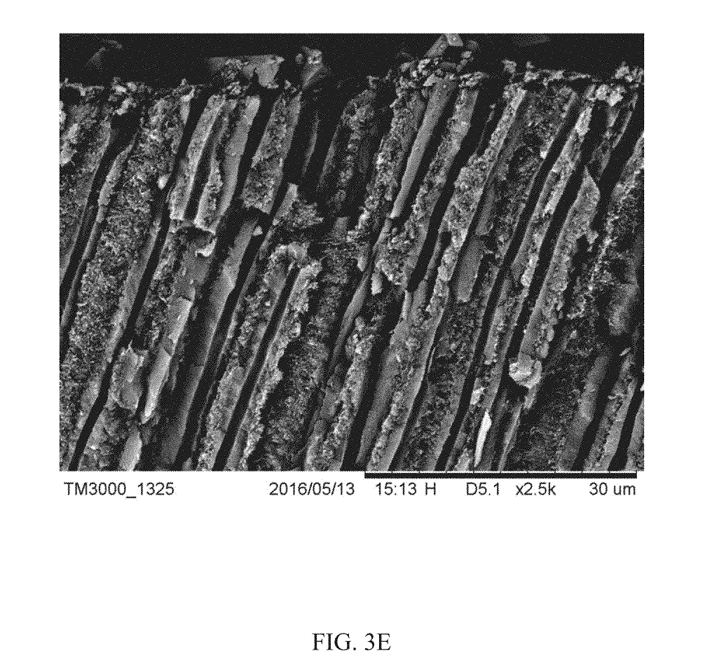

FIG. 3A showed the SEM observations of the specimen of the surface of tooth sample treated with Sample 7 for 1 hour. FIGS. 3B-3C respectively showed the SEM observations of each of the specimens of the surface of tooth samples treated with Sample 8 for 1 hour and 1 day. FIGS. 3D-3E respectively showed the SEM observations of each of the specimens of the surface of tooth samples treated with Sample 9 for 1 hour and 1 day. By comparing FIGS. 3B-3C and FIGS. 3D-3E, it was showed that there is no any sealing effect of the formulation having a molar ratio of calcium ion to phosphate ion of 0.2 with a pH value of 4 or 5; however the sealing effect thereof was shown as the formulations of Samples 8 and 9 were reacted with the surfaces of tooth samples for 1 day. Besides, the depths of the precipitates in the dentinal tubules of each of Samples 7-9 after occluding for different period of time were shown in the following Table 8. The results indicated that the formulation having a higher pH value could provide the sealing effective on dentin by increasing reaction time. In other words, the formulation having a lower pH value could seal the dentin quickly.

TABLE 8
The depths of the precipitates in the dentinal tubules of
Samples 7-9 with different reaction time

| | Depth (μm) | Duration (hour) |
|---|---|---|
| Sample 7 | 59 | 1 |
| Sample 8 | X | 1 |
| Sample 8 | 43 | 24 |
| Sample 9 | X | 1 |
| Sample 9 | 44 | 24 |

To examine the sealing effect of the formulations of Samples 10-13 with a molar ratio of metal ion to phosphate ion (i.e., Mg/P, Sr/P, Zn/P or Zr/P) of 0.2 and a pH value of 2 or 3, each of the formulation of Samples 10-13 was applied on the surfaces of tooth samples, allowing it to react for 1 day. Then, the depth of the precipitates in the dentinal tubules in each of the specimens was observed by SEM.

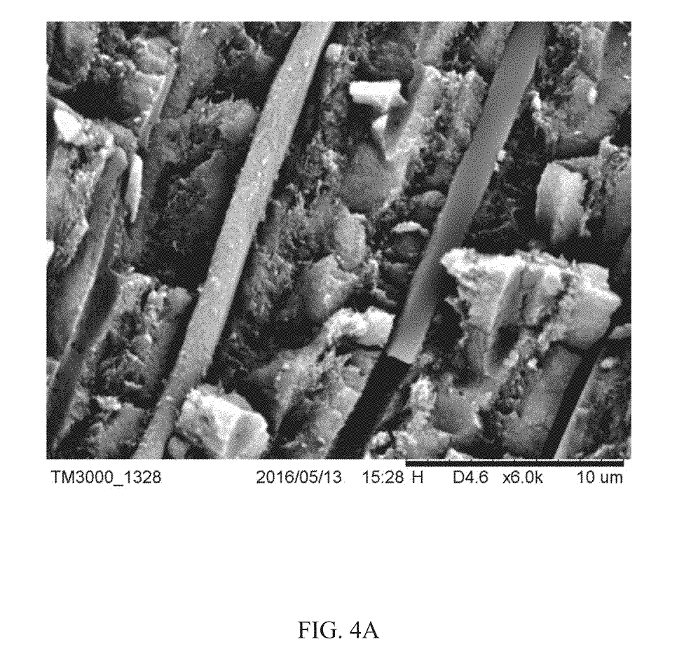

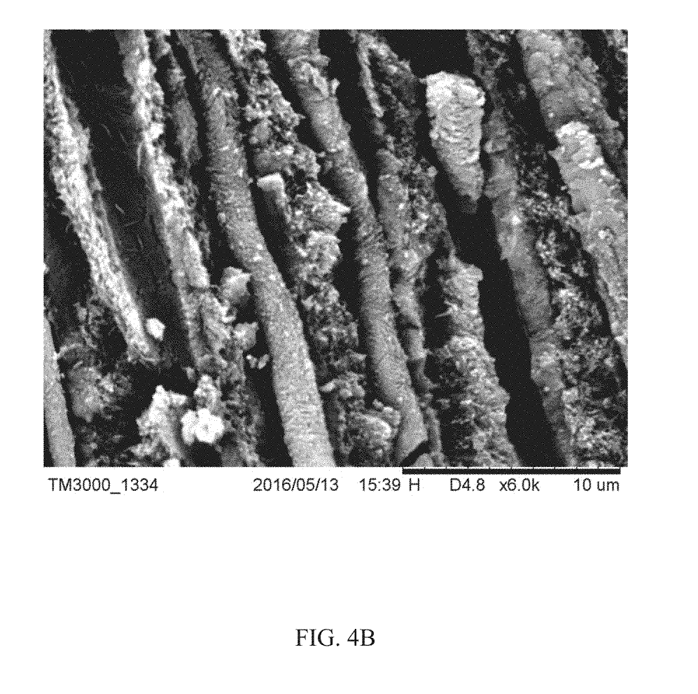

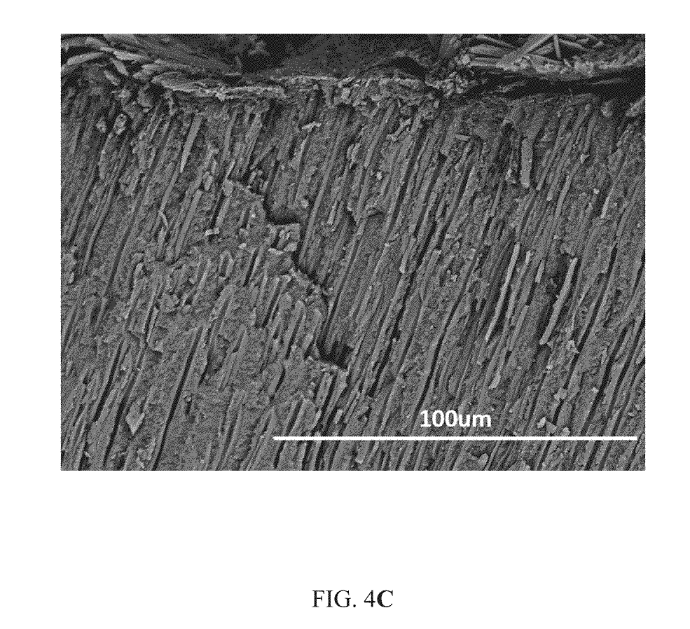

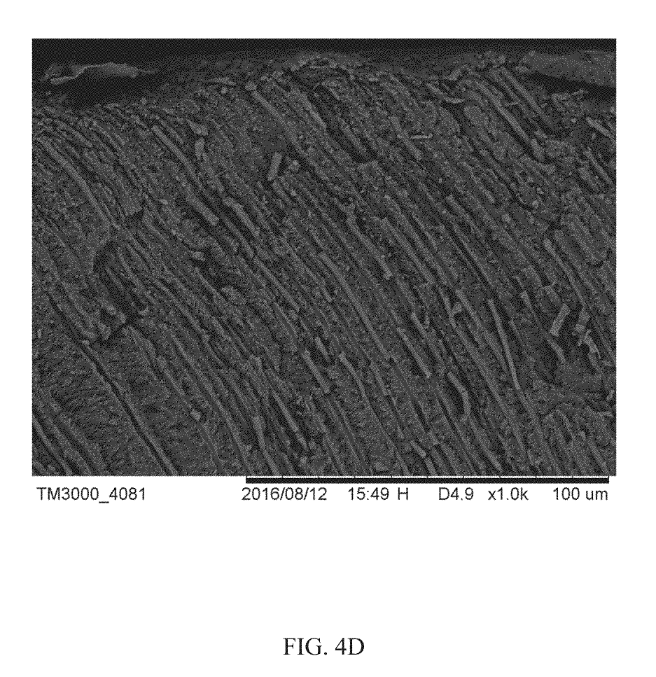

FIGS. 4A-4D respectively showed the SEM observations of each of the specimens of the surface of tooth samples treated with Samples 10-13 for 1 day, and the depths of the precipitates in the dentinal tubules of Samples 10-13 after occluding were shown in Table 9. The results indicated that the formulation having the metal ion selected from Mg, Sr, Zn and Zr could also provide the sealing effective on dentin. In other words, metal ion within the formulation could be selected from alkaline earth metals.

TABLE 9
The depths of the precipitates in the dentinal tubules of Samples 10-13

| | depth (μm) |
|---|---|
| Sample 10 | 255 |
| Sample 11 | 235 |
| Sample 12 | 110 |
| Sample 13 | 206 |

To examine the sealing effect of the formulations of Samples 14-18 with a molar ratio of calcium ion to phosphate ion of 0.2, a pH value of between 2-3 and an additive (zinc phosphate, methyl cellulose, carbomer, titanium dioxide or calcium phosphate), each of the formulation of Samples 14-18 was applied on the surfaces of tooth samples, allowing it to react for 1 day. Then, the depth of the precipitates in the dentinal tubules in each of the specimens was observed by SEM.

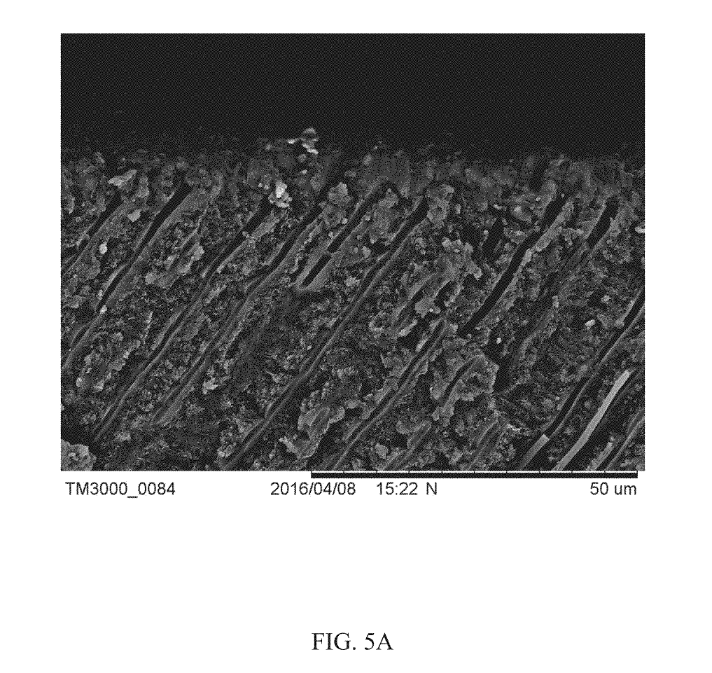

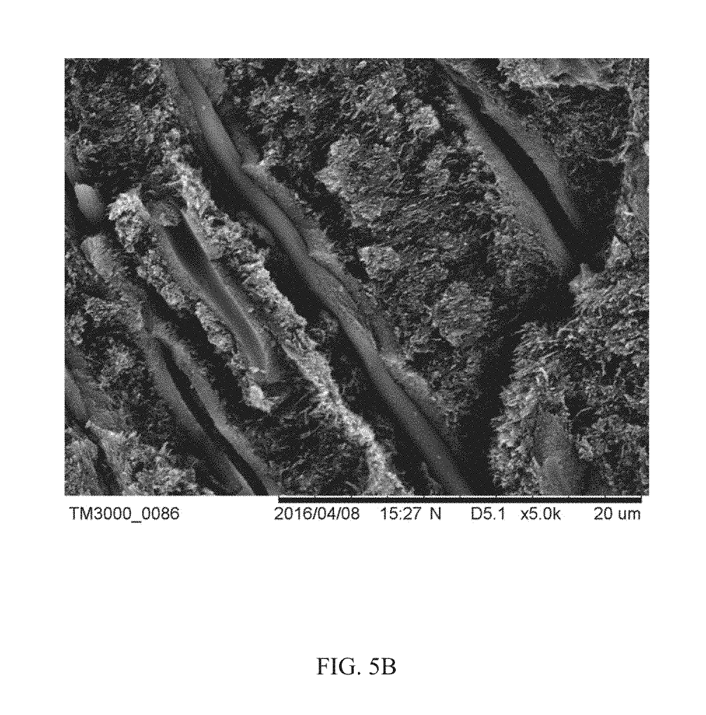

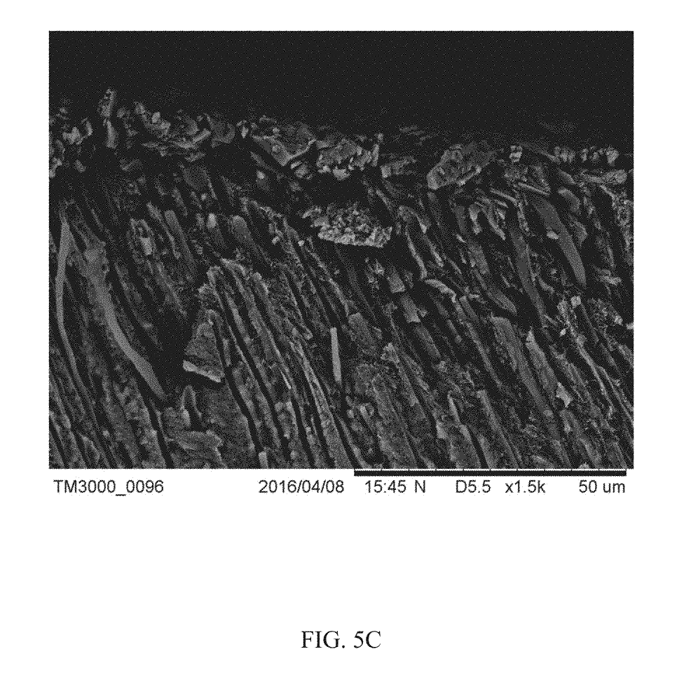

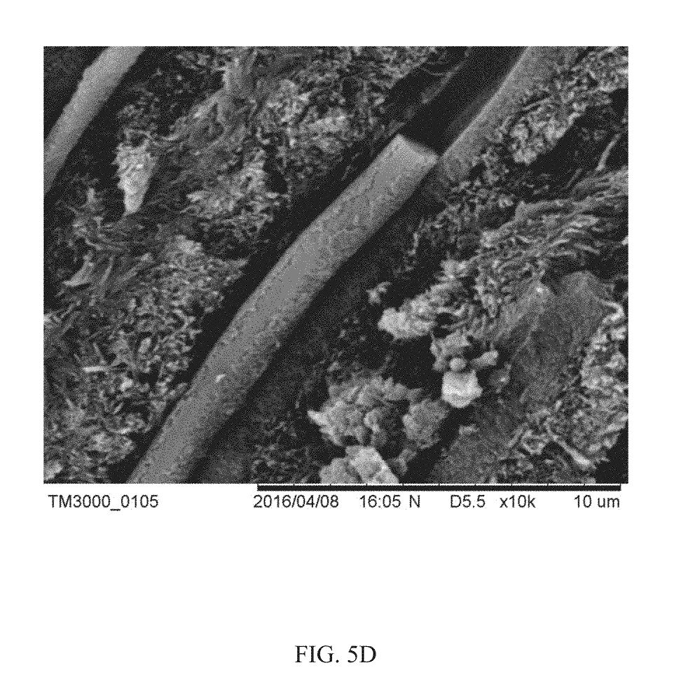

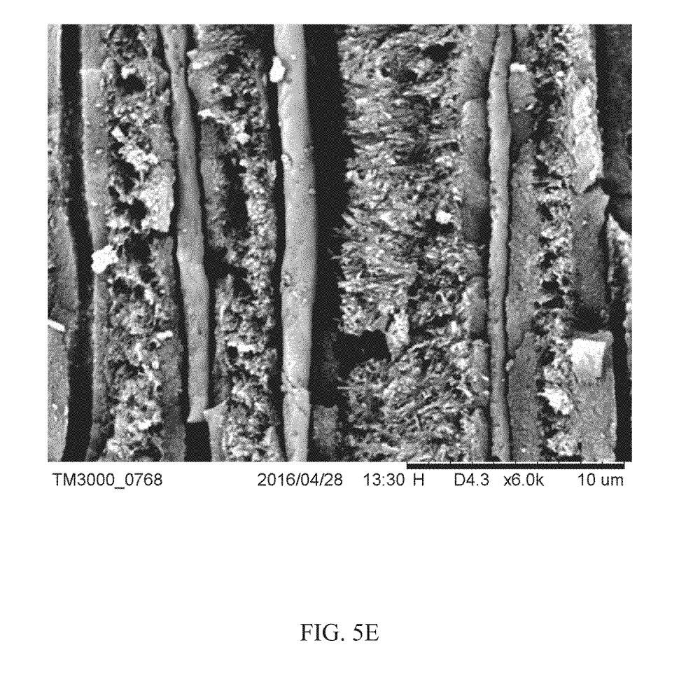

FIGS. 5A-5E respectively showed the SEM observations of the specimen of the surface of tooth sample treated with Samples 14-18 for 1 day, and the depths of the precipitates in the dentinal tubules of each of Samples 14-18 were shown in the following Table 10. The results indicated that the formulations containing different additives could also provide the sealing effective on dentin.

TABLE 10
The depths of the precipitates in the dentinal tubules of Samples 14-18.

| | depth (μm) |
|---|---|
| Sample 14 | 90 |
| Sample 15 | 245 |
| Sample 16 | 165 |
| Sample 17 | 306 |
| Sample 18 | 98 |

To examine the sealing effect of the formulation of Sample 19 in a form of orabase, the formulation of Sample 19 was applied on the surfaces of tooth samples, allowing it to react for 1 day. Then, the depth of the precipitates in the dentinal tubules in each of the specimens was observed by SEM.

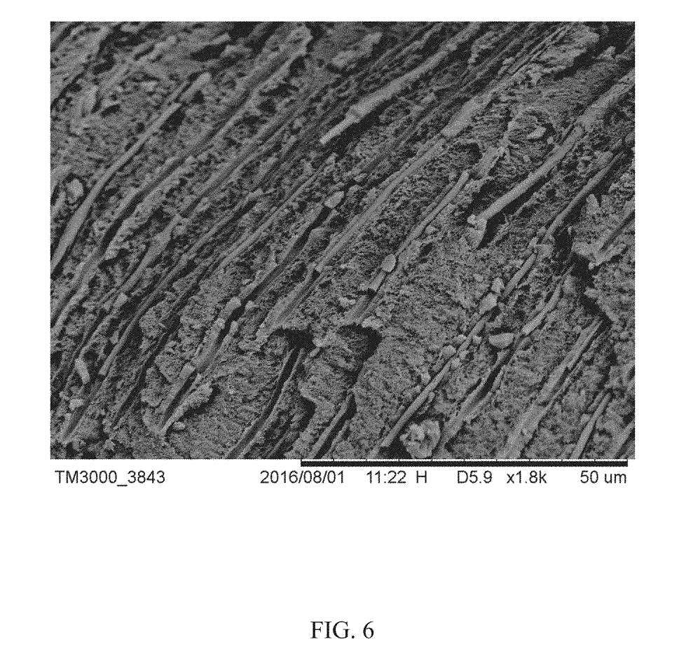

FIG. 6 shows the SEM observation of the specimen of the surface of tooth sample treated with Sample 19 for 1 day, and the depth of the precipitate in the dentinal tubules of Sample 19 was more than 100 μm. The result indicated that the formulation in a form of orabase could also provide the sealing effective on dentin.

### Test Example 2: Permeability Test on Dentinal Tubules In Vitro

To determine whether the phosphate-containing drug comprised in the formulation of the present disclosure enters into the dentinal tubules, the quantity and the permeability of the phosphate-containing drug released from the formulation of the present disclosure coated on the dentin were evaluated by high-performance liquid chromatography (HPLC) assay. Before HPLC, the formulation comprising the phosphate-containing drug was applied on the surfaces of tooth samples, allowing it to react for 1 day. Then, the substance penetrated through the dentinal tubules was collected to be analyzed. The assays for the formulation of Samples 20 and 21 containing the phosphate-containing drug were respectively performed as follows.

### Assay I

Preparation of Standard 1:

Betamethasone sodium phosphate RS (reference standard) was dissolved in a mixture of methanol and water (3:2), and then diluted to a concentration of about 0.16 mg/ml.

Preparation of Sample 20:

KH₂PO₄ was ground and mixed well with SiO₂ as thickening agent, and then CaCl₂ powder and betamethasone sodium phosphate were added thereto and mixed well to form a solid material. The solid material was then mixed with water to obtain Sample 20. The amounts of SiO₂, KH₂PO₄, CaCl₂ powder and betamethasone, as well as water in Sample 20 were shown in the following Table 11.

TABLE 11
Contents of Sample 20

| | | | | Beta- | | | |
|---|---|---|---|---|---|---|---|
| | | | | methasone | | | |
| | | | CaCl₂ | sodium | | | |
| | SiO₂ | KH₂PO₄ | powder | phosphate | Water | Ca/P | pH |
| | (g) | (g) | (g) | (g) | (g) | ratio | value |
| Sample | 0.0742 | 0.21 | 0.03425 | 0.1992 | 0.415 | 0.4-0.6 | 2-3 |
| 20 | | | | | | | |

Preparation of Mobile Phase:

A mixture of methanol and 0.07 M anhydrous monobasic potassium phosphate (3:2) was degassed.

High-performance liquid chromatography was performed by separately injecting equal volumes (about 20 μl) of Standard 1 and Sample 20 into a 4.0 mm×30 cm column that contains packing L1, and the flow rate was about 1.5 ml/min. Betamethasone sodium phosphate RS was detected with a 254 nm detector. The content of betamethasone sodium phosphate in Sample 20 was calculated as following formula:

(As/At)×(weight of Standard 1/weight of Sample 20)×(50/100)×(0.7/volume of Sample 20)×F×100

As represents average area of betamethasone sodium phosphate in Standard; At represents area of betamethasone sodium phosphate in Sample, and F represents content of the standards in percentage.

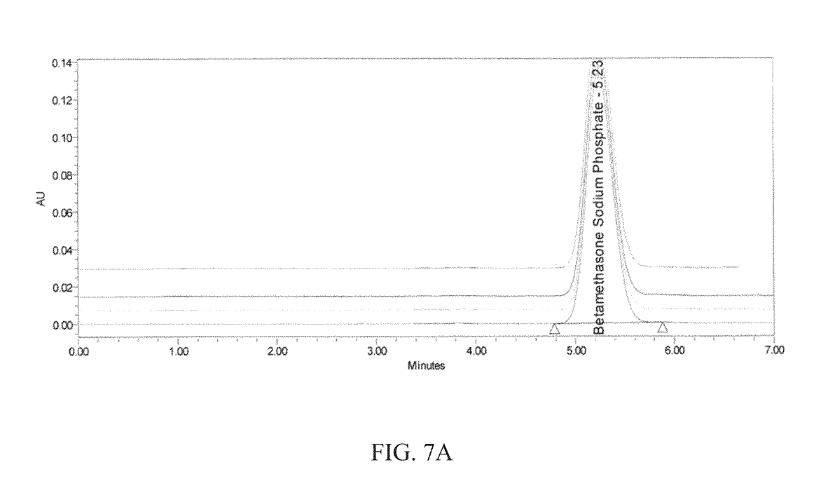

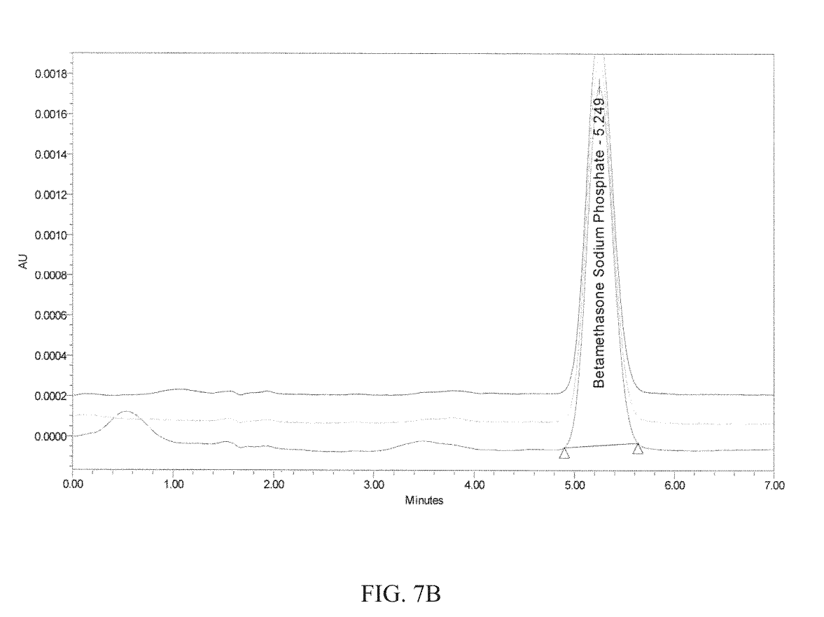

As shown in FIGS. 7A and 7B, the result showed that the retention time of Standard 1 and Sample 20 was identical. Besides, The value of “As” was 32214.3; the value “At” was 2488643.6; the value of “weight of Standard 1” was 16.1; the value of “weight of Sample 20” was 200.2; the value of “volume of Sample 20” was 0.5, and the value of “F” was 98.9%. After calculation, the permeability of the dentin specimen of Sample 20 was about 0.0721%. That is to say, the formulation of Sample 20 entered into the dentinal tubules, and thus provided good sealing effect on dentin.

### Assay II

Preparation of Sample 21:

KH₂PO₄ was ground and mixed well with SiO₂ as thickening agent, and then CaCl₂ powder and clindamycin phosphate were added thereto and mixed well to form a solid material. The solid material was then mixed with water to obtain Sample 21. The amounts of SiO₂, KH₂PO₄, CaCl₂ powder and clindamycin phosphate, as well as water were shown in the following Table 12.

TABLE 12
Contents of Sample 21

| | | | CaCl₂ | Clindamycin | | | |
|---|---|---|---|---|---|---|---|
| | SiO₂ | KH₂PO₄ | powder | phosphate | Water | Ca/P | pH |
| | (g) | (g) | (g) | (g) | (g) | ratio | value |
| Sample | 0.0742 | 0.21 | 0.03425 | 0.1992 | 0.415 | 0.4-0.6 | 2-3 |
| 21 | | | | | | | |

Preparation of Buffer Solution:

14 ml of phosphoric acid was added to 4000 mL of HPLC grade water, and then added 10 ml of ammonium hydroxide thereto, and finally adjust with ammonium hydroxide to a pH value of 3.90±0.05.

Preparation of Organic Solution:

Acetonitrile was mixed with methanol in a ratio of 9:1.

Preparation of Diluent:

The buffer solution was mixed with the organic solution in a ratio of 8:2.

Preparation of Solution I:

The buffer solution was mixed with the organic solution in a ratio of 92:8.

Preparation of Solution II:

The buffer solution was mixed with the organic solution in a ratio of 52:48.

Preparation of Mobile Phase:

The solution I was mixed with the solution II in a ratio to prepare the mobile phase.

Preparation of Standard 2:

42.7 mg of USP clindamycin phosphate RS was dissolved in 20 ml of dilute to prepare the Standard 2.

HPLC was performed by separately injecting equal volumes (about 20 μl) of Standard 2 and Sample 21 into a 4.6 mm×25 cm column that contains packing L7, and the flow rate was about 1.2 ml/min Clindamycin phosphate was detected with a 214 nm detector. The quantity, in percentage, of clindamycin in the portion of clindamycin phosphate was calculated as following formula:

(As/At)×(weight of Standard 2/weight of Sample 21)×(100/20)×(50/1)×(50/5)×(0.7/volume of Sample 21)×F

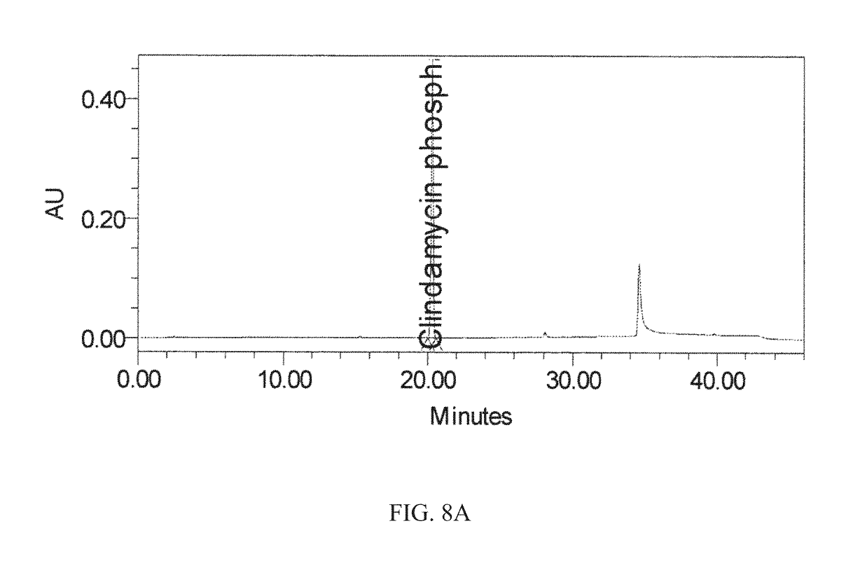

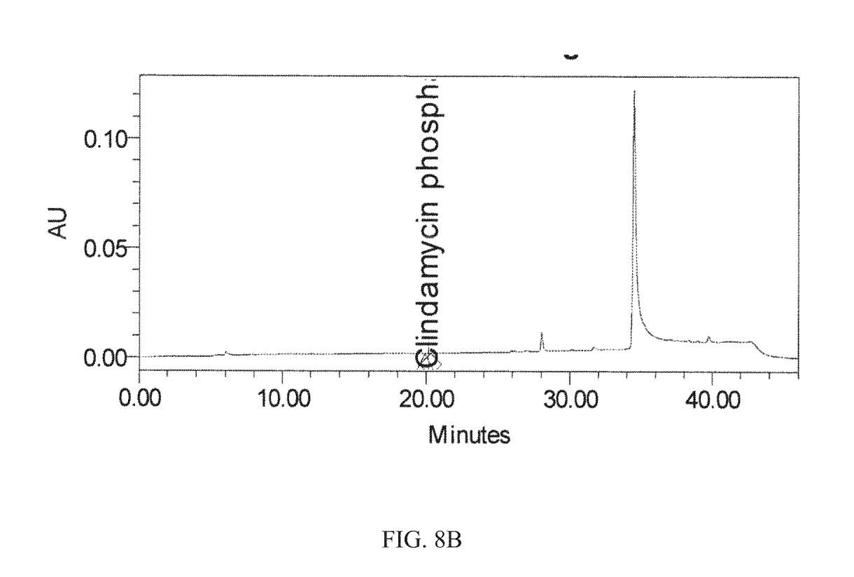

As shown in FIGS. 8A and 8B, the result showed that the retention time of Standard 2 and Sample 21 was identical. Besides, The value of “As” was 692.1; the value “At” was 4311011.2; the value of “volume of Sample 21” was 0.5 the value of “weight of Standard 2” was 42.7; the value of “weight of Sample 21” was 166.42, and the value of “F” was 83.4%. After calculation, the permeability of the dentin specimen of Sample 21 was about 12.02%. That is to say, the formulation of Sample 21 entered into the dentinal tubules, and thus provided good sealing effect on dentin.

### Test Example 3: Sealing Effect of Dentinal Tubules In Vitro of the Formulation Containing Phosphate-Containing Drug

In this example, a formulation of Sample 22 containing betamethasone sodium phosphate was provided. Preparation of the formulation of Sample 22 was similar to those described in the above examples. The Ca/P ratio and pH value of the formulation of Sample 22 were 0.25 and 4, respectively. To examine the sealing effect of the formulation containing the phosphate-containing drug, the formulation of Sample 22 was applied on the surfaces of tooth samples, allowing it to react for 1 day. Then, the depth of the precipitates in the dentinal tubules in each of the specimens was observed by SEM.

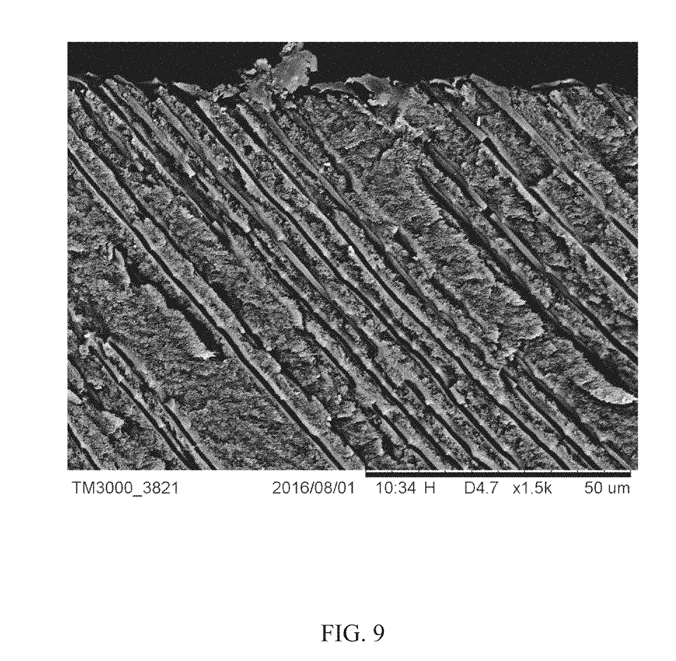

FIG. 9 shows the SEM observation of the specimen of the surface of tooth sample treated with Sample 22 for 1 day, and the depth of the precipitate in the dentinal tubules of Sample 20 was more than 100 μm. Moreover, the permeability of the dentin specimen of Sample 22 was about 1.45%. The result indicated that the formulation containing phosphate-containing drug could also provide the sealing effective on dentin.

The disclosure has been described using exemplary preferred embodiments in detail in the above. However, it is to be understood that the scope of the disclosure is not limited to the disclosed embodiments. On the contrary, it is intended to cover various modifications and similar rearrangement. The scope of the claims therefore should be accorded the broadest interpretation so as to encompass all such modifications and similar arrangements.

1. A method for treating or preventing an exposed dentinal tubule-associated symptom or disease, comprising administering a non-aqueous formulation to dentinal tubules of a subject in need thereof, wherein the non-aqueous formulation comprises:
a source of a metal ion; and
a source of a phosphate ion,
wherein the metal ion is chosen from Mg, Ca, Sr, Zn, Zr or any combination thereof, and a molar ratio of the metal ion to the phosphate ion in the formulation is between about 0.01 and about 1.0, and wherein the metal ion and the phosphate ion form a precipitate in the dentinal tubules of the subject.

2. The method of claim 1, wherein the molar ratio of the metal ion to the phosphate ion in the formulation is between about 0.1 and about 1.

3. The method of claim 1, wherein the molar ratio of the metal ion to the phosphate ion in the formulation is between about 0.2 and about 1.

4. The method of claim 1, wherein the molar ratio of the metal ion to the phosphate ion in the formulation is between about 0.2 and about 0.6.

5. The method of claim 1, wherein the formulation is used in combination with water or saliva to form a mixture having a pH value between about 2 and about 6.

6. The method of claim 5, wherein the pH value of the mixture is between about 2.0 and about 5.5.

7. The method of claim 5, wherein the pH value of the mixture is between about 2.0 and about 5.0.

8. The method of claim 5, wherein the pH value of the mixture is between about 2.0 and about 4.0.

9. The method of claim 5, wherein the pH value of the mixture is between about 3.0 and about 4.0.

10. The method of claim 1, wherein the metal ion is selected from the group consisting of a magnesium ion, a calcium ion, and a strontium ion.

11. The method of claim 1, wherein the source of the metal ion is selected from the group consisting of carbonates, acetates, lactates, citrates, chlorides, oxides, nitrates and hydroxides.

12. The method of claim 1, wherein the source of the phosphate ion is at least one selected from the group consisting of disodium hydrogen phosphate, dipotassium hydrogen phosphate, lithium dihydrogen phosphate, sodium dihydrogen phosphate, potassium dihydrogen phosphate, trisodium phosphate, tripotassium phosphate, ammonium phosphate and a phosphate-containing drug.

13. The method of claim 12, wherein the phosphate-containing drug is selected from the group consisting of tetracycline phosphate complex, oleadomycin phosphate, codeine phosphate, estramustine phosphate, primaquine phosphate, dimemorfan phosphate, pyrldoxal phosphate, pyridoxal phosphate, piperazine phosphate, clindamycin phosphate, sodium phosphate, dexamethasone sodium phosphate, oseltamivir phosphate, benproperine phosphate, prednisolone sodium phosphate, betamethasone sodium phosphate, chloroquine phosphate, disopyramide phosphate, etoposide phosphate, fludarabine phosphate, histamine phosphate, hydrocortisone sodium phosphate, sodium biphosphate, ruxolitinib phosphate, sitagliptin phosphate, anileridine phosphate, sonidegib phosphate, oritavancin diphosphate, tedizolid phosphate, antazoline phosphate, estramustine phosphate, toceranib phosphate and chromic phosphate P32.

14. The method of claim 1, further comprising an additive selected from the group consisting of a thickening agent, an adhesive, an excipient, a stabilizer, an emulsifier, a humectant, and a combination thereof.

15. The method of claim 14, wherein the thickening agent is selected from the group consisting of methyl cellulose, hydroxyethyl cellulose, carbomer, titanium dioxide, zinc phosphate, zinc oxide, silicon dioxide, silicoaluminate, aluminum oxide and calcium phosphate.

16. The method of claim 14, wherein the adhesive is selected from the group consisting of acacia, alginate, alginic acid, candelilla wax, carnuba wax, corn starch, copolyvidone, ethyl cellulose, gelatin, glyceryl behenate, hydroxyl propyl cellulose, hydroxyl propyl methyl cellulose, hypromellose, lactose hydrous, lactose anhydrous, lactose monohydrate, lactose spray dried, methyl cellulose, povidone, polyvinylpyrrolidone, polyethylene oxide, potato starch, starch pregelatinized, starch, sodium starch and sodium carboxy methyl cellulose.

17. The method of claim 14, wherein the excipient is pectin, eudragit or a combination thereof.

18. The method of claim 1, being in a form of a powder, a paste, a flake, a gel, a soft gel, a gum, a semi-solid, a slurry, a patch, an emulsion, a glue, a buccal tablet, a pill, a film, a cream, an aerosol, or an orabase.

19. (canceled)

20. The method of claim 1, wherein the exposed dentinal tubule-associated symptom is selected from the group consisting of dentin hypersensitivity, crack tooth syndrome, enamel loss, dentin loss and postoperative hypersensitivity.

21. The method of claim 1, wherein the exposed dentinal tubule-associated disease is selected from the group consisting of dental caries, root caries, tooth fracture, root fracture, cervical abrasion, tooth wearing, root perforation, radicular cyst, apicitis, pulpitis, periapical periodontitis, pulp necrosis and exposed dentinal associated pulp disease.

22. The method of claim 1,
wherein the non-aqueous formulation
is administered to the dentinal tubules of the subject by smearing, pasting, attaching or brushing.

23. A method for treating or preventing dentin hypersensitivity, comprising administering a non-aqueous formulation to dentinal tubules of a subject in need thereof, wherein the non-aqueous formulation comprises:
a source of a metal ion; and
a source of a phosphate ion,
wherein the metal ion is chosen from Mg, Ca, Sr, Zn, Zr or any combination thereof, and a molar ratio of the metal ion to the phosphate ion in the formulation is between about 0.01 and about 1.0, and wherein the metal ion and the phosphate ion form a precipitate in the dentinal tubules.
